(19) United States
(12) Patent Application Publication
(10) Pub. No.: US 2002/0168782 A1
(43) Pub. Date: Nov. 14, 2002

(54) Detection of allergen-specific IgE

(76) Inventors: Catherine A. McCall, Boulder, CO (US); Uma Mahesh Babu, Fort Collins, CO (US); Steven V. Radecki, Fort Collins, CO (US)

Correspondence Address: Heska Corporation, 1613 Prospect Parkway, Fort Collins, CO, 80525, US

(21) Appl. No.: 10/038,519

(22) Filed: Jan. 3, 2002

Related U.S. Application Data
(60) Provisional application No. 60/325,812, filed on Sep. 28, 2001.
(60) Provisional application No. 60/259,450, filed on Jan. 3, 2001.

(51) Int. Cl. G01N 33/543

(52) U.S. Cl. 436/518

(57) ABSTRACT

This invention relates to point-of-care devices and methods to screen animals with suspected IgE-mediated allergic disease. The present invention provides a simple and rapid preliminary immunoassay screen, in which a defined mixture of clinically relevant allergens is used to capture allergen-specific IgE present in a sample, followed by a second binding reagent that selectively binds IgE. The present invention further provides methods for prescribing immunotherapy treatment in animals having IgE-mediated diseases. Devices and kits useful for carrying out the methods of the invention are also provided.

### CROSS REFERENCE TO RELATED APPLICATIONS

This application claims priority to U.S. Provisional Patent Application Serial No. 60/325,812, filed on Sep. 28, 2001 and U.S. Provisional Patent Application Serial No. 60/259,450, filed on Jan. 3, 2001, both entitled “IN-CLINIC TEST FOR THE DETECTION OF ALLERGEN-SPECIFIC IgE” and are incorporated herein by reference in their entirety.

### FIELD OF THE INVENTION

This invention relates to point-of-care test (POCT) devices and methods to screen animals showing signs of allergy or atopic disease. More particularly, the immunological devices and methods of the invention use a defined mixture of clinically relevant allergens to detect allergen-specific IgE in samples obtained from such animals.

### BACKGROUND OF THE INVENTION

The cross-linking of mast cell-bound allergen-specific IgE to allergens induces Type I allergic diseases such as atopic dermatitis and atopic asthma. Diseases related to allergy and atopy affect a significant percentage of the population, including up to 20% of humans, and are increasing every year.

Allergy-related illness is not restricted to humans, and can be a major problem in veterinary practice as well. A number of other diseases may present with symptoms similar to those of allergy/atopy, thus a definitive diagnosis may be difficult. A definitive diagnosis relies upon a thorough history, careful review of clinical signs, and in vitro and in vivo diagnostic tests.

Immunoassay procedures have provided sensitive diagnostic tools for the in vitro detection of a variety of allergens of clinical significance. These procedures use allergens and IgE specific detection reagents to measure allergen-specific antibodies in the serum of the patient. These processes characteristically require an incubation period with both the allergens and other reagents and, as a result, may be time consuming. It is not unusual for an assay procedure to require up to 24 hours to complete. Furthermore, the need to adhere to time incubation steps and multiple washings with measured reagents has largely limited these procedures to large hospital and reference clinical laboratories where highly trained personnel and sophisticated equipment are available to perform the assays. Additionally, a certain percentage of atopic patients are allergic to allergens not present in the standard allergen-panels used by reference labs. In these cases, detailed testing fails to reveal the aggravating allergen(s) resulting in these patients incurring unnecessary medical expenses.

Presently there is an unmet need for a simple and rapid procedure for conducting immunoassays for allergen specific IgE which can be used in the physician's or veterinarian's office. The present invention satisfies this need and provides related advantages as well.

### SUMMARY OF THE INVENTION

The present invention provides rapid and simple tests to aid in the diagnosis of allergy in animals, including, but not limited to, humans, dogs, cats and horses. More particularly, the devices and methods of the invention screen for allergen-specific IgE in a sample using immunological reactions. Such samples can be obtained from animal bodily fluids that contain IgE, such as, for example, urine, mucosal secretions, milk, whole blood or components thereof including plasma, serum and the like.

The devices of the present invention include a first member containing a porous member, such as a membrane or filter, having a sample receiving area. The porous member can be spotted with at least one mixture of allergens (in one spot or multiple spots) and at least one control spot. The control spot or spots can be a positive control, such as purified IgE, and/or a procedural control, such as biotinylated bovine serum albumin (BSA), for example. The mixture of allergens can be spotted either within the sample receiving area or in a separate area of the porous member.

The device can optionally contain an absorbent member in capillary communication with the porous first member to induce the flow of a liquid through the porous member without the use of external means. The absorbent member is selected to have a capillary pore size to induce flow of liquids through the porous member when the hydrostatic pressure of the sample and subsequent reagents are insufficient to induce flow through the porous member.

The device also includes a support member to support or house the first porous member. The absorbent member can also provide the means to support the first member.

Methods for detecting the presence of allergen-specific IgE in a sample use simple devices of the present invention, small volumes, and do not require lengthy incubation steps. Such methods are accomplished by:

(a) adding the sample to the sample receiving area of a device of the present invention, whereby at least one allergen in the mixture of allergens spotted on the porous member of the device binds to allergen-specific IgE if present in the sample;

(b) contacting the mixture of allergens with a reagent that selectively binds the allergen-specific IgE; and

(c) detecting bound allergen-specific IgE if present in the sample.

Useful mixtures of allergens for spotting onto the porous member can contain, but are not limited to, one or more types of allergens selected from the following: cat allergens, dog allergens, flea saliva proteins, house dust mite allergens, storage dust mite allergens, Japanese Cedar pollen, ragweed allergens, ryegrass allergens, meadow fescue, orchard grass, Bermuda grass, sheep sorrel, Kochia, Russian thistle, yellow dock, timothy grass, redtop, birch and blue grass allergens.

Useful reagents that selectively bind the allergen-specific IgE in a sample include, for example, anti-IgE antibodies or IgE receptors or mimetopes of such molecules. Such reagents can be labeled with a detectable marker or a detectable marker can be separately added that binds or otherwise associates with the reagent such that detection is possible.

The present invention accordingly provides low cost point-of-care (i.e., in clinic or field) tests that can be performed simply and quickly in the physician's or veterinarian's office. The rapid results available with the methods and devices of the present invention permit the physician or veterinarian to quickly assess the need for further diagnostic work and/or treatment plans. Further diagnostic work may include submission of a sample to a central diagnostic laboratory for testing to determine the exact allergens for which the animal is positive, and for which immunotherapy would be appropriate.

Thus, the present invention further provides methods for prescribing immunotherapy treatment of an animal having an allergic disease. Briefly, the methods are accomplished by:

(a) obtaining a sample from the animal;

(b) analyzing the sample according to the methods of the present invention to detect the presence of allergen-specific IgE in the sample, wherein the presence of allergen-specific IgE identifies the sample as a positive sample;

(c) analyzing the positive sample to identify one or more allergens responsible for an allergic disease; and

(d) selecting an appropriate immunotherapy to treat the allergic disease.

The present invention further provides novel reagents, including anti-IgE antibodies, and kits containing devices and ancillary reagents for conducting the immunoassays.

### DETAILED DESCRIPTION OF THE INVENTION

The present invention is generally directed to POCT devices and immunoassay methods for the rapid detection of allergen-specific IgE. The devices and methods of the present invention are useful as preliminary aids in the diagnosis of allergy in animals.

The immunoassay methods of the present invention use POCT devices that include a first porous member and a support member. Particularly suitable devices include the following elements: (1) a porous solid surface to which a defined mixture of allergens can be spotted in one or more spots; (2) a sample receiving area for adding the sample to be assayed; (3) a means for drawing liquids through the porous surface; and (4) an area for displaying the results. In certain embodiments, one element may perform the functions of one or more of the other elements. For example, the sample receiving area may be the same area or a different area from where the defined mixture of allergens have been spotted.

The porous first member can be made of any suitable material to which the mixture of allergens can be bound or affixed, while sufficiently porous to allow the flow of liquids to pass through or by the affixed mixture of allergens. Suitable materials include membranes, filters and the like. Such membranes and filters can be made from a variety of materials, which include but are not limited to, for example, plastic, glass, gel, celluloid, paper, fabric, polystyrene, nylon, nitrocellulose, agarose, cotton, and PVDF (poly-vinylidene-fluoride). The mixture of allergens can be spotted (i.e. affixed) on the porous member by any means known to those skilled in the art or as described in the Examples below.

One or more controls can also spotted on the porous member to ensure the proper operation of the immunoassay. Preferably, the control spot is an area on the device, which comes into contact with the assay reagents and which contains an agent which serves to indicate binding of IgE-detection reagent has occurred. Agents useful as positive controls include, for example, purified polyclonal or monoclonal IgE, IgE receptors or active fragments thereof, anti-IgE immunoglobulin, anti-IgG immunoglobulin, protein A, mimetopes of any of the above molecules or any other molecule resulting in binding of the IgE-detecting reagent to the porous member. As used herein, the term “mimetope” refers to any compound able to mimic the ability of anti-IgE antibody to bind to IgE. An example of a mimetope can be a peptide that has been modified to decrease its susceptibility to degradation but which still retains the ability to bind the IgE-detecting reagent. Other examples of mimetopes include, but are not limited to, carbohydrate-based compounds, lipid-based compounds, nucleic acid-based compounds, natural organic compounds, synthetically derived organic compounds, anti-idiotypic antibodies and/or catalytic antibodies, or fragments thereof. A mimetope can be obtained by, for example, screening libraries of synthetic compounds for compounds capable of binding to the IgE-detecting reagent. Procedural controls known to those skilled in the art can also be spotted on the porous member, including, for example, biotinylated BSA. The porous first member further contains a sample receiving area. As noted previously, the sample receiving area can be the same area where the mixture of allergens are spotted or can be in a different location on the porous member depending on the desired format. A pre-filter can optionally be used for samples that are preferably filtered prior to introducing the filtered sample to the sample receiving area, including, for example, whole blood to obtain serum. Such samples can be filtered separately from the device of the present invention in a pre-filter device or the pre-filter device can be seated above the sample receiving area and, if desired, the pre-filter can be removed and disposed after the sample has been pre-filtered.

The devices of the present invention optionally contain an absorbent member having capillary pathways connected to the porous member to permit a liquid to traverse the absorbent member. Accordingly, the absorbent member is in capillary communication with the porous member and has capillary pores sized to induce the flow of liquid through the first porous member without the use of external means. The absorbent member is particularly useful when the hydrostatic pressure of the sample and subsequent reagents used in the assay are insufficient to induce flow through the first member. In addition, the absorbent member can also provide support for the first member.

Those skilled in the art can readily identify suitable materials useful as absorbent members and other support members. Thus, the devices of the present invention can be of any standard format known to those skilled in the art, including, for example, a lateral flow device (as described in U.S. Pat. No. 5,073,484 or U.S. Pat. No. 5,654,162, both incorporated herein by reference), a flow-through device (as described in U.S. Pat. No. 4,727,019, incorporated herein by reference), or any modifications of such devices readily known or determined by those skilled in the art.

The methods of the present invention for detecting the presence of allergen-specific IgE in a sample obtained from an animal are generally accomplished by first adding a sample to the sample receiving area of a device as described above. After the sample has come in contact with the test spot where the mixture of allergens has been spotted on the porous member, a reagent that selectively binds allergen-specific IgE is added and allowed to come in contact with the test spot. If present in the sample, the bound allergen-specific IgE is then detected.

As used herein, the term “animal” refers to any mammal that is capable of having an allergic disease caused by the presence of deleterious amounts of allergen-specific IgE. Such animals preferably include, but are not limited to, humans, dogs, cats and horses. As used herein, a cat refers to any member of the cat family (i.e., Felidae), including domestic cats, wild cats and zoo cats. Examples of cats include, but are not limited to, domestic cats, lions, tigers, leopards, panthers, cougars, bobcats, lynx, jaguars, cheetahs, and servals. A preferred cat to test is a domestic cat. As used herein, a dog refers to any member of the family Canidae, including, but not limited to, domestic dogs, wild dogs, foxes, wolves, jackals, and coyotes and other members of the family Canidae. As used herein, a horse refers to an equid. An equid is a hoofed mammal and includes, but is not limited to, domestic horses and wild horses, such as, horses, asses, donkeys, and zebras. Preferred horses to test include domestic horses, including race horses.

Samples to be assayed can be obtained from bodily fluids of such animals. Any bodily fluid that contains allergen-specific IgE can be used, including, for example, urine, mucosal secretions, milk, whole blood and components thereof, such as serum and plasma. Further examples of bodily fluids may be found in U.S. Pat. No. 6,309,832 which is hereby incorporated by reference. A bodily fluid can be used undiluted as the sample or can be diluted as desired in a suitable diluent before applying the sample to the sample receiving area. Those skilled in the art can readily determine the appropriate sample to be used in a particular assay.

The allergens that are selected in the defined mixture of allergens that is deposited on the porous member are selected such that a minimum number of spots, preferably one spot, will identify a great majority of patients with IgE-mediated allergic disease. Preferably, the defined mixture of allergens will be capable of identifying at least 50%, 55%, 60%, 65%, 70%, 75%, 80%, 85%, 90%, 95%, 99% and 100% of patients with IgE-mediated allergic disease. Those skilled in the art can readily determine a defined mixture of allergens capable of identifying a desired percent of the relevant population. For example, if the desired target is 90% of all human patients with IgE-mediated allergic disease, those skilled in the art would select a mixture of allergens that are collectively known to cause 90% of all IgE-mediated allergies in humans. Ideally, the mixture of allergens is chosen such that the minimum number of allergens allows the identification of the maximum number of patients. Various embodiments of the present invention may have different mixtures of allergens based on the patient population to be tested.

The allergens may be in the form of extracts, purified allergenic proteins from extracts or from raw materials such as mites or pollens, recombinant allergens, or combinations of the above. Suitable allergens for use in the defined mixture include, but are not limited to, cat allergens (U.S. Pat. Nos. 5,328,991; 6,019,972; 6,048,962; and 6,120,769); dog allergens (U.S. Pat. No. 5,843,672); flea saliva proteins (U.S. Pat. Nos. 5,646,115; 5,795,862; and 5,840,695); house dust mite allergens (U.S. Pat. Nos. 5,433,948; 5,552,142; 5,770,202; 5,773,002; 5,820,862; 5,986,526; 5,972,352; 6,060,057; 6,071,522; 6,077,518; 6,086,897; 6,132,734; and 6,077,517); Japanese Cedar pollen (U.S. Pat. No. 6,090,386); Johnson grass allergens (U.S. Pat. Nos. 5,480,972; 5,691,167; and 5,736,149); ragweed allergens (U.S. Pat. No. 5,698,204); ryegrass allergens (U.S. Pat. Nos. 5,710,126; 5,840,316; and 5,965,455); meadow fescue, orchard grass, Bermuda grass, sheep sorrel, Kochia, Russian thistle, yellow dock, timothy grass, redtop, and blue grass allergens. Additional allergens may be found in U.S. Pat. No. 6,309,832. The above U.S. patents are hereby incorporated by reference.

In the methods of the present invention, after the sample comes in contact with the spotted mixture of allergens in order to allow any allergen-specific IgE to bind to one or more of the allergens in the mixture, a reagent that selectively binds allergen-specific IgE is then added and allowed to bind to any bound allergen-specific IgE. The term “second binding reagent” is used interchangeably with “reagent that selectively binds allergen-specific IgE.” The detection of allergen-specific IgE bound to the second binding reagent is indicative of the presence of allergen-specific IgE in the sample.

As used herein, a reagent that “selectively binds allergen-specific IgE” means that it preferentially binds IgE as compared to IgG or another class of immunoglobulin such that the second binding reagent does not bind to IgG or another class of immunoglobulin in an amount that substantially affects the results of the immunoassay. For example, a suitable second binding agent binds IgE to a significantly greater extent (has a substantially higher affinity for IgE) than IgG. In a preferred embodiment, the second binding agent binds IgE with a specificity similar to that exhibited by the human IgE receptor alpha chain protein. In another embodiment, the second binding agent fails to bind IgG under conditions in which known anti-IgE antibodies bind IgE. Binding of immunoglobulin by binding agents of the present invention can be measured using procedures known to those skilled in the art, for example , the ELISA procedures described herein. In a particularly preferred embodiment, the second binding agent binds IgG with an absorbence value, as determined by ELISA, of less than 300, 250, 225, 200, 175, 150, 125 or 100. Guidance for selecting suitable binding agents is provided in the Examples below.

The second binding reagent may be a polyclonal or monoclonal antibody that selectively binds IgE, the Fcε receptor 1 (FcεR1) (as described in U.S. Pat. No. 5,945,294, which is incorporated herein by reference), IgE-binding allergens or any molecule which selectively binds to IgE. One example of such an antibody is an anti-IgE antibody that only recognizes antibodies which bind to mast cells and basophils. Preferred antibodies of the present invention have the characteristics of a mAb produced using human FcεRIα-affinity purified IgE as described herein.

In a preferred embodiment, the second binding reagent is a monoclonal antibody that selectively binds to an IgE receptor-binding region or domain. Examples of such antibodies include, but are not limited to, for example, monoclonal antibodies H-219, H-195, H-202, H-203 and H-207. Species-specific antibodies and receptors are particularly useful for detecting allergen-specific IgE in samples obtained from that species. For example, canine anti-IgE antibodies are useful as the second binding reagent in assays for detecting allergen-specific IgE in sample obtained from a dog.

In a preferred embodiment, the second binding reagent is monoclonal antibody that selectively binds to canine IgE. Examples of such antibodies include, but are not limited to, for example, monoclonal antibodies H-219, H-220, H-189, H-190, H-195, H-197, H-200, H-202, H-203, H-206 and H-207. In another preferred embodiment, the second binding reagent is monoclonal antibody that selectively binds to feline IgE. Examples of such antibodies include, but are not limited to, for example, monoclonal antibodies H-99, H-100, H-101, H-102, H-106. In another preferred embodiment, the second binding reagent is monoclonal antibody that binds to both canine and feline IgE. Examples of such antibodies include, but are not limited to, for example, monoclonal antibodies H-201, H-192, H-198, H-199, H-208. In another preferred embodiment, the second binding reagent is monoclonal antibody that selectively binds to equine IgE. Examples of such antibodies include, but are not limited to, for example, monoclonal antibody H-357.

In a further preferred embodiment, the second binding reagent is an IgE receptor. The term “IgE receptor” includes the full length receptor and active fragment thereof. As used herein, the term “active fragment thereof” means fragments of the full length receptor having binding sites that are capable of binding to the allergen-specific IgE captured by the allergen(s) contained in the defined mixture.

In one embodiment, the second binding reagent can be directly conjugated to, or labeled with, an indicator agent. In an alternative embodiment, an indicator agent capable of binding to or associating with the second binding reagent can be separately added and detected. Those skilled in the art can readily identify suitable detection means for use in the present methods.

Any indicator or detection reagent such as enzymes, fluorescent molecules, radiolabels, or other detectable marker known to those skilled in the art can be used. Enzymes are particularly useful as indicator agents. If the label is an enzyme, after washing to remove unbound receptor, a solution of the enzyme substrate is added. If the device has bound to it the allergen specific IgE, then the IgE will have bound to it a portion of labeled receptor. The enzyme will cause the substrate to react and generate, if properly selected, a new, and easily detectable enzymatic-reaction end product. A particularly useful end product is a chromophore which results in a visual color change. If a label other than an enzyme is used, the procedure can be varied. Fluorescence on the porous member can be measured by known methods if a fluorescing label is used. If a radionuclide label, such as ¹²⁵I is used, the porous member can be removed and counted.

The addition of the sample, second binding reagent and any desired washing buffers can be preceded by brief incubation periods to permit more extensive binding of the allergen-specific IgE in the sample to increase the sensitivity of the assay. However, in the preferred methods of the present invention, such incubation steps are either unnecessary or are very brief, i.e., generally on the order of 60 seconds or less. The flow of solutions containing the sample or second binding reagent results in a substantially faster rate of binding than is observed in the absence of flow. Accordingly, the total time required for performing the test is about 5 minutes or less, and preferably less than about 3 minutes.

The present invention also provides methods of prescribing immunotherapy treatment in animals having IgE-mediated allergic disease. Such methods include obtaining a sample from an animal having symptoms of allergic disease, analyzing the sample according to the immunoassays methods described above to detect the presence of allergen-specific IgE. If allergen-specific IgE is detected in the sample, the positive sample is then further analyzed to identify one or more allergens responsible for the allergic disease and an appropriate immunotherapy is then selected for the animal. Further analysis of a positive sample to identify the specific allergen or allergens responsible for the allergic disease can be accomplished by any means known to those skilled in the art, including, for example, by in vitro immunoassays conducted for example in a reference laboratory or by intradermal tests (e.g. conventional skin tests). Once the specific allergen or allergens have been identified, clinicians skilled in the art can determine the appropriate immunotherapy for the particular patient.

Kits for performing the methods of the present invention are also provided. In addition to the devices of the present invention, the kits of the present invention include reagents used in the methods of the present invention. Such reagents include wash buffers and detection reagents. Preferred detection reagents of the invention include monoclonal antibodies that specifically bind canine, feline, equine, and human IgE.

The following examples are provided for the purposes of illustration, and are not intended to limit the scope of the present invention.

### EXAMPLE 1

This example describes how the allergens for the screens are selected, and how the devices are prepared.

The methods of the present invention use carefully defined mixtures of allergens. The mixtures are selected to identify most allergic individuals with a minimum allergen panel.

Data from the assignee's US diagnostic laboratories was analyzed to identify allergens to which many dogs were allergic. The point of care test was designed to detect 70 to 90% of animals that give positive results in the complete panel at the diagnostic laboratories, while keeping the total number of allergens in the point of care test small.

A total of more than two thousand samples was analyzed in several different sets. The samples were tested in the full panel of allergens and approximately 80% tested positive to at least one allergen in the panel. (An animal that tests negative to the complete panel may be an animal that is allergic to an allergen not in the panel, or may not be allergic at all, but rather have another disease with symptoms that resemble allergy).

Of those 80%, approximately 60% are positive to either flea or dust mites. To add an additional 15% of animals, other important allergens may be added. Candidate additional allergens would be Meadow Fescue, Orchard Grass, red Cedar, yellow dock, timothy grass, redtop, and blue grass. A different set of allergens may be desired in different geographic regions and/or climates, and/or for different animals (the above data is for dogs). The desired set of allergens is again determined by analysis of allergen specific IgE detected in a standard or reference laboratory tests.

Data from other regions was analyzed in a similar manner. Nearly 800 dog sera were analyzed for antibodies to indoor allergens. House dust mite was the most common allergen, followed by storage mites and flea saliva. A point of care device with house dust mite, storage mite, and flea saliva could detect 98% of the animals that are positive in the indoor panel.

More than 500 dog sera were analyzed for United Kingdom general pollens; 72% of the dogs were positive to cocksfoot. The next most prevalent pollen allergens were alder, English Plantain, Lamb's quarters, and Sheep Sorrel. A point of care device with allergens from house dust mite, storage mite, and cocksfoot or highly related grasses, could detect 91% of the animals that are positive in the complete panel.

Approximately 30 dog sera were analyzed for Central Europe panel; 100% of the dogs were positive to house dust mite or storage mites. A point of care device with house dust mite and storage mite could detect 100% of the animals that are positive in the complete panel.

Approximately 56 dog sera were analyzed for Northern Europe panel; 65% of the dogs were positive to house dust mite. The next most prevalent allergens were storage mites, followed by red top and velvet grass. A point of care device with house dust mite and storage mite could detect 98% of the animals that are positive in the complete panel.

Approximately 122 dog sera were analyzed for United Kingdom extended pollens; 58% of the dogs were positive to Meadow fescue. The next most prevalent allergen was Grand fir. A point of care device with Meadow fescue could detect 58% of the animals that are positive in the complete panel.

Approximately 104 dog sera were analyzed for Mediterranean panel; 59% of the dogs were positive to house dust mite. The next most prevalent allergens were storage mites and flea saliva. A point of care device with house dust mite, storage mite, and flea saliva could detect 97% of the animals that are positive in the complete panel.

To determine the correct allergens to use in the devices and methods of the invention it is also necessary to assess such allergens in a point of care format, to determine that the sera will still test positive when the allergens are present as a mixture, rather than as an isolated sample.

In one embodiment of the present invention, the point of care device is prepared by spotting the porous member with one mixture of allergens comprising meadow fescue, dust mite, and flea saliva allergens. Each of these allergens may be present as crude extract, purified extract, and/or genetically engineered and recombinantly expressed pure proteins. The allergens extracts or recombinant proteins are spotted onto the porous member at approximately 1,000 to 500,000 PNU/ml; or 10 μg/ml to 10 mg/ml, depending upon the particular allergen. The exact concentration required for a test must be determined experimentally, but one can begin with a concentration similar to that used in an ELISA format with the same allergen or extract. The allergens are spotted onto the porous member with spotting buffer without BSA (TRIS 6.05 g/l; NaCl 8.5 g/l; NaN₃ 20 mg/l; and sucrose 10 g/l).

A spot of canine IgE is added at a different location as positive control. This is spotted at 1 μg/ml to 10 mg/ml, preferably about 5-10 μg/ml in spotting buffer with BSA (TRIS 6.05 g/l; NaCl 8.5 g/l, NaN₃ 20 mg/l, sucrose 10 g/l, and BSA 1 g/l).

In another embodiment of the invention, there are two test spots on the device, one with meadow fescue and house dust mite, and the other with flea saliva proteins, in addition to the control spot of canine IgE.

### EXAMPLE 2

This example describes the production of mouse anti-canine IgE monoclonal antibodies (mAb) essentially by the method of Kohler and Milstein (Kohler and Milstein, (1975), Nature 256:495-497), which is herein incorporated by reference.

Canine IgE was prepared by pooling canine serum and fractionating by ammonium sulfate precipitation. The fraction from 30-55% saturation was collected and passed through a Protein G column. The flow through fraction was applied to a rHuman IgE Receptor Affiprep (Biorad) column. Retained IgE was recovered from the column with high pH elution. A polybuffer Exchanger column further purified IgE. Fractions eluting at pH 5.7 to 4.95 were pooled.

Two Balb/c mice were immunized in the footpad with 30 μg canine IgE suspended in phosphate buffered saline (PBS) and Freund's complete adjuvant. A boost of 30 μg canine IgE was given in PBS/Freund's incomplete adjuvant in the footpad on day 14. Sera was tested for presence of anti-canine IgE antibodies at day 21. The mouse exhibiting the highest titer against canine IgE by ELISA was boosted with 5 μg canine IgE in PBS, intravenously on day 42. Spleenoctyes were harvested three days later, and fused with mouse SP2/0 myeloma cells at mid-log growth phase using PEG.

Cells were cultured in RPMI medium containing 20% fetal bovine serum, 10% thymocyte conditioned media, 2 mM L-glutamine, 1 mM sodium pyruvate, and 60 μM β-mercaptoethanol. Hybrids were selected by adding 100 μM hypoxanthine, 10 μM thymidine and 0.4 μM aminopterin. Wells containing hybridoma colonies were tested for specific mAb production against canine IgE, IgG, and IgM by ELISA. Antigens were coated at 5 ng/well. Wells positive to canine IgE were expanded and dilution cloned by limiting dilution until stable mAb secreting cell lines were identified.

Monoclonal antibody is either supplied in a crude form as tissue culture supernatant, ascites or bioreactor supernatant. Specific mAb can be purified using affinity chromatography or ion exchange chromatography from any of these fluid types.

Twenty antibodies reactive to canine IgE epsilon chain by ELISA were generated. All mAbs recognize canine IgE and not canine IgG or IgM by ELISA. Of these, fifteen recognize the 71 kDa epsilon chain by Western blots except three (H-198, 199, and 206) and two are yet to be tested (H-192 and 204). Three Western blots patterns emerged using these mAbs to probe canine IgE; one set of mAbs (H-195, 197, and 208) recognize the 71 kDa epsilon chain of canine IgE only; a second set of mAbs (H-189, 190, 191, 193, 203, 207, 219 and 220) recognize the 71 kDa epsilon chain of canine IgE and a band running right above the dye front (at about 6 kDa); and the third set of mAbs (H-200, 201, 202, and 205) recognize the 71 kDa epsilon chain of canine IgE and a broad diffuse band at 55 kDa. This third set of mAbs do not recognize canine IgG gamma chain, suggesting that the extra band is not a contaminant of dog IgG. This interesting binding domain appears to have some relationship with the canine IgE epsilon chain. All but one mAb (H-204) were evaluated, and five of these mAbs (H-191, 192, 198, 199, and 208) recognized feline IgE by ELISA even better than canine IgE, and in some cases reactivity with canine IgE was not even detectable. One mAb (H-201) recognized feline IgE equally well as canine IgE.

Antibodies were grouped into 9 categories based on ELISA reactivity patterns. The highest titered mAb from each group was purified and screened as a canine IgE detection POCT candidate (H-189, 199, 200, 201, 204, 205, 206, 207, and 208). From these mAbs, H-200, 201, and 207 were chosen. Further testing with these three mAbs indicated that H-207 was the preferred mAb to be utilized in the POCT.

The antibodies mentioned above can be utilized in various assay configurations to detect various levels of canine IgE present in complex samples such as dog sera, PBMCs or possibly even blood. They work well in combination with the human FcεRIα to calculate total serum IgE. With appropriate standards, dog IgE levels could easily be measured and compared to various stages of immune stimulation, allergen exposure, etc. Additional applications of anti-canine IgE mAbs include application to simplifying purification of canine IgE and/or canine IgE immune complexes by affinity chromatography methods. Use of mAbs specific to epsilon chain would easily and more readily remove dog IgE from sample sources such as serum as compared to conventional chromatography methods. Other uses include monitoring separation of intact canine IgE, epsilon chain during purification processes. Flow cytometry studies could be performed by using these antibodies to map cell surface IgE and to check intact canine IgE, epsilon chain during purification processes. Flow cytometry studies could be performed by using these antibodies to map cell surface IgE and to check the validity of FcεRIα based results. In addition, the anti-canine epsilon chain mAbs may be used for immunotherapy for allergic dogs.

Mouse anti-feline IgE mAbs, mouse anti-equine IgE mAbs, and mouse anti-human IgE mAbs are made in the same manner as above. The mAbs may also be prepared in animals other than mouse by standard techniques for preparation of monoclonal antibodies for the above cited uses.

### EXAMPLE 3

This Example discloses the ability of the anti-canine IgE monoclonal antibodies to bind antigen-specific, canine or feline IgE, as measured by solid phase ELISA, using the following protocol:

The wells of a microtiter plate were coated with either Der F protein (30 Protein Nitrogen Units (PNU)/well) (Center Labs, Port Washington, N.Y.), flea saliva antigen (FSA) (100 ng/well) (prepared as described in U.S. Pat. No. 5,646,115) or canine IgG (1 μg/well) (Jackson ImmunoResearch Labs, West Grove, Pa.), diluted in CBC buffer (50 mM carbonate, pH 9.6) and the plate stored, covered, overnight at 4° C. (Wells coated with IgG serve to measure the level of non-specific immunoglobulin binding.) The following day, excess fluid was removed from the wells, the plates blotted dry, and 200 μl of Assay buffer (4% fetal calf serum in PBS containing 0.05% Tween-20) were added to each well. Following a 60 minute incubation at room temperature (RT), the wells were washed four times using Wash buffer (PBS containing 0.05% Tween-20), and 100 μl of canine or feline serum (diluted 1:20 in Assay buffer) were added to each well. Some wells (labeled ‘Heated Serum’) received serum which had been heated at 56° C. for one hour prior to use. (This procedure is known to denature the IgE-Fc receptor binding region which allows one to determine which antibodies bind IgE in or near this region) The plates were incubated overnight at 4° C., after which the wells were washed four times using Wash buffer, and 100 μl of supernatant from anti-canine IgE hybridoma cells (prepared as described in Example 2), diluted in Assay buffer as indicated, were added to each well. The plates were incubated for 2 hours at room temperature after which, the wells were washed four times using Wash buffer and 100 μl of HRP-conjugated, donkey, anti-mouse IgG (Jackson ImmunoResearch Labs, West Grove, Pa.), diluted 1:2000 in Assay buffer, were added to each well. After incubating at RT for one hour, the wells were washed four times using Wash buffer and 100 μl of TMB Peroxidase Substrate System (KPL Labs, Gaithersburg, Md.) were added to each well. The plates were incubated at RT until color developed within the wells (approximately ten minutes), at which time the reaction was stopped by adding 100 μl of 1.0M phosphoric acid to each well. Background levels of non-specific binding were measured by preparing wells as described above but without adding canine or feline serum. Using an ELISA plate reader, the optical density of each well was measured at 450 nM, the results of which are shown in Table 1. The background level of absorbence was subtracted from all of the values shown in Table 1. Values over 100 are considered to be positive.

TABLE 1

| | Dog Serum | | | | Cat Serum | | | |
|---|---|---|---|---|---|---|---|---|
| | | DerF | | | Flea Saliva Ag | | | Dog |
| mAb | MAb | | Heated | Serum | | Heated | Serum | IgG |
| ID | Dilution | Serum | Serum | (−) | Serum | Serum | (−) | Serum |
| H-219 | 1:250 | 3249 | 479 | 40 | 5 | 22 | 5 | 455 |
| | 1:500 | 3028 | 369 | | 4 | 4 | | |
| | 1:1000 | 2819 | 270 | | 10 | 10 | | |
| H-220 | 1:250 | 49 | 1533 | 0 | 7 | 6 | 4 | 316 |
| | 1:500 | 42 | 1234 | | 8 | 4 | | |
| | 1:1000 | 60 | 870 | | 7 | 6 | | |
| H189 | 1:250 | 39 | 1838 | 2 | 7 | 6 | 8 | 473 |
| | 1:500 | 37 | 1714 | | 3 | 1 | | |
| | 1:1000 | 42 | 1465 | | 2 | 3 | | |
| H-190 | 1:250 | 2783 | 2970 | 0 | 65 | 21 | 12 | 1192 |
| | 1:500 | 2227 | 2798 | | 34 | 10 | | |
| | 1:1000 | 1578 | 2426 | | 24 | 7 | | |
| H-191 | 1:250 | 35 | 57 | 0 | 3630 | 2636 | 0 | 22 |
| | 1:500 | 46 | 55 | | 3517 | 2351 | | |
| | 1:1000 | 44 | 60 | | 3245 | 1714 | | |
| H-192 | 1:250 | 55 | 62 | 8 | 3418 | 2926 | 35 | 64 |
| | 1:500 | 44 | 52 | | 3071 | 2273 | | |
| | 1:1000 | 48 | 71 | | 2595 | 1808 | | |
| H-193 | 1:250 | 72 | 1572 | 0 | 13 | 7 | 9 | 368 |
| | 1:500 | 61 | 1306 | | 11 | 14 | | |
| | 1:1000 | 71 | 972 | | 8 | 13 | | |
| H-195 | 1:250 | 3752 | 2027 | 0 | 26 | 30 | 22 | 1260 |
| | 1:500 | 3604 | 1477 | | 18 | 21 | | |
| | 1:1000 | 3424 | 974 | | 21 | 27 | | |
| H-197 | 1:250 | 1953 | 2882 | 0 | 28 | 12 | 6 | 902 |
| | 1:500 | 1294 | 2415 | | 17 | 11 | | |
| | 1:1000 | 739 | 1779 | | 14 | 16 | | |
| H-198 | 1:250 | 120 | 60 | 0 | 3818 | 2923 | 1 | 14 |
| | 1:500 | 81 | 44 | | 3803 | 2841 | | |
| | 1:1000 | 79 | 45 | | 3714 | 2779 | | |
| H-199 | 1:250 | 60 | 50 | 1 | 3108 | 2502 | 0 | 85 |
| | 1:500 | 44 | 47 | | 2977 | 2242 | | |
| | 1:1000 | 43 | 47 | | 2736 | 1959 | | |
| H-200 | 1:250 | 3781 | 2420 | 4 | 29 | 33 | 28 | 1355 |
| | 1:500 | 3744 | 1897 | | 15 | 17 | | |
| | 1:1000 | 3532 | 1436 | 11 | 11 | | | |
| H-201 | 1:250 | 3606 | 2538 | 1 | 3695 | 3061 | 26 | 1782 |
| | 1:500 | 3473 | 2195 | | 3557 | 2480 | | |
| | 1:1000 | 3223 | 1904 | | 3446 | 2705 | | |
| H-202 | 1:250 | 3697 | 1892 | 0 | 20 | 17 | 19 | 1170 |
| | 1:500 | 3826 | 1744 | | 15 | 11 | | |
| | 1:1000 | 3620 | 1343 | | 22 | 9 | | |
| H-203 | 1:250 | 3803 | 2425 | 0 | 69 | 69 | 68 | 1986 |
| | 1:500 | 3603 | 2024 | | 39 | 42 | | |
| | 1:1000 | 3424 | 1478 | | 32 | 30 | | |
| H-205 | 1:250 | 67 | 3483 | 0 | 12 | 6 | 9 | 472 |
| | 1:500 | 66 | 1245 | | 6 | 3 | | |
| | 1:1000 | 68 | 752 | | 8 | 10 | | |
| H-206 | 1:250 | 1972 | 1446 | 0 | 3 | 18 | 8 | 472 |
| | 1:500 | 1615 | 1284 | | 4 | 3 | | |
| | 1:1000 | 1156 | 972 | | 9 | 11 | | |
| H-207 | 1:250 | 3393 | 720 | 4 | 12 | 11 | 12 | 663 |
| | 1:500 | 3085 | 546 | | 8 | 6 | | |
| | 1:1000 | 2617 | 353 | | 11 | 10 | | |
| H-208 | 1:250 | 105 | 51 | 1 | 3053 | 2939 | 5 | 22 |
| | 1:500 | 101 | 67 | | 2672 | 2797 | | |
| | 1:1000 | 122 | 59 | | 2184 | 2488 | | |

### EXAMPLE 4

The ability of the anti-canine IgE monoclonal antibodies to bind canine or feline IgE bound by immobilized human Fc-epsilon receptor I-alpha chain, was tested using the following capture-assay protocol.

The wells of a microtiter plate were coated with the extracellular domain of human Fc-epsilon receptor I-alpha chain protein (huFcεRIα) (prepared as described in U.S. Pat. No. 5,945,294 ) (42.5 ng/well) in CBC buffer, and the plate incubated, covered, overnight at 4° C. Additionally, Sseveral wells were also coated with heat-treated canine IgG (Jackson ImmunoResearch Labs, West Grove, Pa.) (1 μg/well) to measure the background level of non-specific immunoglobulin binding. It is believed that heat treatment specifically destroys the activity of contaminating IgE.

The following day, excess fluid was removed from the wells, 200 μl of Assay buffer added to each well and the plate incubated at RT for one hour. The wells were washed four times using Wash buffer, and purified canine or feline IgE (prepared as described in Example 2) was added to each well (50 ng/well in 100 μl Assay buffer). Following a two hour incubation at RT, the wells were washed four times using Wash buffer and 100 μl of anti-canine IgE hybridoma cell supernatant (prepared as described in Example 2), diluted as indicated in Assay buffer, were added to each well. The plate was incubated at RT for one hour, the wells washed four times using Wash buffer, and 100 μl of HRP-conjugated, donkey, anti-mouse IgG diluted 1:2000 in Assay buffer, were added to each well. The plate was incubated for one hour at RT after which, the wells were washed four times using Wash buffer, and 100 μl of TMB Peroxidase Substrate System (KPL Labs, Gaithersburg, Md.) were added to each well. The plate was incubated at RT until color developed within the wells (approximately 15 minutes) at which time the reaction was stopped by adding 100 μl of 1.0M phosphoric acid to each well. The optical density of each well was measured at 450 nM using an ELISA plate reader, the results of which are shown in Table 2. Background absorbence was measured by preparing some wells as described above but without any mAb's and background levels were subtracted from all values listed in Table 1. Values over 100 are considered positive.

TABLE 2

| | H-219 | | | H-220 | | | H-189 | | |
|---|---|---|---|---|---|---|---|---|---|
| mAb | Dog | Cat | Dog | Dog | Cat | Dog | Dog | Cat | Dog |
| Dilution | IgE | IgE | IgG | IgE | IgE | IgG | IgE | IgE | IgG |
| 1:100 | 2897 | 11 | 77 | 26 | 5 | 1 | 30 | 4 | 2 |
| 1:300 | 2960 | 6 | 38 | 11 | 5 | 0 | 22 | 0 | 0 |
| 1:1000 | 2679 | 5 | 17 | 8 | 0 | 0 | 17 | 4 | 0 |
| | H-190 | | | H-191 | | | H-192 | | |
| mAb | Dog | Cat | Dog | Dog | Cat | Dog | Dog | Cat | Dog |
| Dilution | IgE | IgE | IgG | IgE | IgE | IgG | IgE | IgE | IgG |
| 1:100 | 3985 | 152 | 108 | 15 | 576 | 0 | 2726 | 4200 | 0 |
| 1:300 | 3062 | 53 | 67 | 10 | 353 | 0 | 1199 | 4200 | 10 |
| 1:1000 | 2444 | 17 | 22 | 3 | 137 | 0 | 759 | 3713 | 6 |
| | H-193 | | | H-195 | | | H-197 | | |
| mAb | Dog | Cat | Dog | Dog | Cat | Dog | Dog | Cat | Dog |
| Dilution | IgE | IgE | IgG | IgE | IgE | IgG | IgE | IgE | IgG |
| 1:100 | 24 | 5 | 4 | 4000 | 16 | 127 | 2570 | 78 | 67 |
| 1:300 | 16 | 5 | 0 | 2851 | 11 | 75 | 3132 | 32 | 35 |
| 1:1000 | 9 | 2 | 0 | 2069 | 5 | 25 | 2789 | 14 | 14 |
| | H-198 | | | H-199 | | | H-200 | | |
| mAb | Dog | Cat | Dog | Dog | Cat | Dog | Dog | Cat | Dog |
| Dilution | IgE | IgE | IgG | IgE | IgE | IgG | IgE | IgE | IgG |
| 1:100 | 2871 | 4200 | 3 | 1240 | 3181 | 0 | 3371 | 35 | 137 |
| 1:300 | 1510 | 4200 | 71 | 1091 | 3344 | 1 | 2434 | 19 | 120 |
| 1:1000 | 1187 | 3865 | 0 | 993 | 3068 | 0 | 2857 | 6 | 41 |
| | H-201 | | | H-202 | | | H-203 | | |
| mAb | Dog | Cat | Dog | Dog | Cat | Dog | Dog | Cat | Dog |
| Dilution | IgE | IgE | IgG | IgE | IgE | IgG | IgE | IgE | IgG |
| 1:100 | 2750 | 3423 | 82 | 3523 | 68 | 107 | 2926 | 15 | 177 |
| 1:300 | 3197 | 3732 | 88 | 2938 | 5 | 85 | 3509 | 22 | 197 |
| 1:1000 | 3387 | 3627 | 71 | 2000 | 3 | 33 | 3704 | 13 | 168 |
| | H-205 | | | H-206 | | | H-207 | | |
| mAb | Dog | Cat | Dog | Dog | Cat | Dog | Dog | Cat | Dog |
| Dilution | IgE | IgE | IgG | IgE | IgE | IgG | IgE | IgE | IgG |
| 1:100 | 27 | 0 | 0 | 2153 | 5 | 19 | 2690 | 6 | 35 |
| 1:300 | 6 | 3 | 2 | 2011 | 6 | 14 | 1925 | 10 | 16 |
| 1:1000 | 5 | 0 | 0 | 1553 | 3 | 4 | 1414 | 0 | 4 |

| | H-208 | | |
|---|---|---|---|
| mAb | Dog | Cat | Dog |
| Dilution | IgE | IgE | IgG |
| 1:100 | 131 | 1880 | 0 |
| 1:300 | 219 | 2099 | 0 |
| 1:1000 | 151 | 1617 | 64 |

### EXAMPLE 5

This example discloses the ability of anti-feline IgE monoclonal antibodies to bind antigen-specific canine or feline IgE. Hybridoma cell lines producing anti-feline IgE were generated using the procedure described in Example 2, with the exception the mice were immunized with feline IgE instead of canine IgE. The ELISA's were performed using the solid phase ELISA protocol outlined in Example 3 and substituting the anti-feline IgE hybridoma supernatants for anti-canine IgE hybridoma supernatants. Additionally, to measure the background level of non-specific immunoglobulin binding, some wells were coated with feline IgG instead of canine IgG. The results of these ELISA's are shown in Table 3. Background levels of absorbence have been subtracted fm all values shown in the table. Values greater than 100 are considered positive.

TABLE 3

| | | | | | | Cat Serum | | | | |
|---|---|---|---|---|---|---|---|---|---|---|
| | | Dog Serum | | | | | | | | Pure |
| | | DerF | | Flea Saliva | | DerF | | Flea Saliva | | Cat |
| | | | Heated | | Heated | | Heated | | Heated | IgG |
| | | Serum | Serum | Serum | Serum | Serum | Serum | Serum | Serum | Serum |
| H-100 | 1:250 | 27 | 43 | 40 | 38 | 2473 | 2794 | 2925 | 2371 | 41 |
| | 1:500 | 30 | 46 | 39 | 41 | 1932 | 2269 | 2605 | 2083 | 72 |
| | 1:1000 | 39 | 50 | 39 | 42 | 1287 | 1582 | 2001 | 1479 | 17 |
| H-101 | 1:250 | 56 | 67 | 46 | 45 | 3577 | 3464 | 3741 | 3287 | 59 |
| | 1:500 | 41 | 63 | 38 | 47 | 3144 | 3042 | 3392 | 2966 | 84 |
| | 1:1000 | 74 | 61 | 39 | 45 | 2474 | 2465 | 2921 | 2416 | 21 |
| H-102 | 1:250 | 60 | 54 | 32 | 43 | 3586 | 3444 | 3628 | 3239 | 33 |
| | 1:500 | 34 | 46 | 27 | 36 | 3188 | 3052 | 3383 | 2984 | 53 |
| | 1:1000 | 30 | 47 | 29 | 39 | 2607 | 2501 | 2935 | 2424 | 28 |
| H-103 | 1:250 | 83 | 64 | 43 | 44 | 30 | 35 | 6 | 25 | 0 |
| | 1:500 | 79 | 72 | 53 | 42 | 3 | 68 | 0 | 5 | 0 |
| | 1:1000 | 48 | 54 | 41 | 48 | 7 | 14 | 1 | 7 | 0 |
| H-106 | 1:250 | 53 | 59 | 43 | 43 | 1268 | 1443 | 1580 | 1002 | 0 |
| | 1:500 | 42 | 74 | 38 | 50 | 861 | 1008 | 1257 | 713 | 5 |
| | 1:1000 | 58 | 52 | 45 | 63 | 450 | 537 | 705 | 433 | 0 |
| H-99 | 1:250 | 110 | 205 | 215 | 261 | 3567 | 4097 | 3632 | 3522 | 4200 |
| | 1:500 | 90 | 113 | 130 | 152 | 3363 | 3962 | 3277 | 3315 | 4200 |
| | 1:1000 | 74 | 107 | 111 | 125 | 3016 | 3814 | 3305 | 2978 | 4089 |
| H-104 | 1:250 | 4200 | 4200 | 4200 | 4200 | 4200 | 4200 | 3895 | 3929 | 4200 |
| | 1:500 | 4200 | 4200 | 4200 | 4200 | 4121 | 4200 | 3755 | 3883 | 4200 |
| | 1:1000 | 4200 | 4200 | 4067 | 4093 | 3932 | 3919 | 3717 | 3746 | 3892 |
| H-107 | 1:250 | 49 | 51 | 30 | 42 | 1418 | 2226 | 217 | 587 | 3975 |
| | 1:500 | 45 | 62 | 15 | 47 | 1225 | 1960 | 172 | 504 | 3808 |
| | 1:1000 | 37 | 76 | 40 | 44 | 879 | 1509 | 143 | 420 | 3140 |

This example demonstrates that all of these mAbs recognize feline IgE. Five out of the eight mAbs are specific for feline IgE whereas two mAb recognizes both feline IgE and IgG and one mAb binds feline IGE, IgG and canine IgE.

### EXAMPLE 6

This example discloses the ability of the anti-feline IgE monoclonal antibodies to bind huFcεRIα-immobilized, canine or feline IgE. This example uses the capture-assay protocol described in Example 4 with the exception that anti-feline IgE hybridoma cell supernatants were used in place of anti-canine IgE hybridoma cell supernatants. The results of this assay are shown below in Table 4.

TABLE 4

| | H-99 | | H-100 | | H-101 | |
|---|---|---|---|---|---|---|
| mAb | Canine | Feline | Canine | Feline | Canine | Feline |
| Dilution | IgE | IgE | IgE | IgE | IgE | IgE |
| 1:00 | 0 | 206 | 24 | 2496 | 8 | 2443 |
| 1:300 | 0 | 186 | 11 | 1591 | 0 | 1381 |
| 1:1000 | 0 | 60 | 11 | 846 | 0 | 1111 |
| | H-102 | | H-103 | | H-104 | |
| mAb | Canine | Feline | Canine | Feline | Canine | Feline |
| Dilution | IgE | IgE | IgE | IgE | IgE | IgE |
| 1:00 | 3 | 2325 | 0 | 0 | 333 | 476 |
| 1:300 | 0 | 1002 | 0 | 0 | 229 | 352 |
| 1:1000 | 6 | 1087 | 0 | 0 | 287 | 169 |

| | H-106 | | H-107 | |
|---|---|---|---|---|
| mAb | Canine | Feline | Canine | Feline |
| Dilution | IgE | IgE | IgE | IgE |
| 1:00 | 9 | 1238 | 0 | 37 |
| 1:300 | 0 | 491 | 0 | 33 |
| 1:1000 | 5 | 245 | 2 | 37 |

### EXAMPLE 7

This example discloses a method for the purification of recombinantly produced equine FcRεRIα (EqFcεIα). Cultures expressing recombinant EqFcRεRIα were produced as described in U.S. Pat. No. 6,057,127, and the supernatants collected and adjusted to a conductivity of 2.2 mv and a pH of 6.0. The sample was applied to a SP-Sepharose Hi-Trap column (Pharmacia, Peapack, N.J.) which had been pre-equilibrated with Buffer A (20 mM Na-PO4, pH 6.0). Following application of the sample, the column was washed with Buffer A and the bound proteins eluted using a step-gradient of 0.1M, 0.15M, 0.2M, 0.3M, 0.4M, 0.5M and 1.0M NaCl in Buffer A. Fractions containing EqFcεRIα, which eluted at 0.2M, were pooled, concentrated, and applied to a Superdex 75 10/30 column which had been equilibrated in PBS. 0.5 ml fractions were collected and analyzed using a Novex 14% SDS-PAGe gel (Invitrogen, Carlsbad, Calif.) according to manufacturers instructions and fractions containing EqFcεRIα were pooled.

### EXAMPLE 8

This example discloses the ability of anti-equine IgE monoclonal antibody H-357 to recognize antigen-bound equine IgE using a solid-phase ELISA. mAb H-357 was produced using the procedure outlined in Example 2 with the exception the mice were immunized with equine IgE instead of canine IgE. H-357 was subsequently shown to be specific for equine IgE. The ELISA's were performed using the following protocol:

The wells of a microtiter plate were coated with the indicated allergen and the plate incubated overnight at 4° C. (All allergens were obtained from Greer Labs, Lenoir, N.C. and coated at 30 PNU/well with the exception of D. farinae which was obtained from Center Labs and coated at 30 allergy units (AU)/well). The following day, excess fluid was removed, 200 μl of Assay buffer added to each well and the plate incubated at room temperature (RT) for one hour. The plate was washed four times using Wash buffer, and 100 μl of equine sera (diluted 1:20 in Assay buffer) added to each well and the plate incubated ON at 4° C. The following day, the plate was washed four times using Wash buffer and either biotinylated anti-equine IgE mAb H357 (10 ng), biotinylated, recombinant human Fc epsilon receptor I alpha chain (huFcεRIα) (13 ng) (prepared as described in U.S. Pat. No. 5,945,294) or biotinylated recombinant equine Fc epsilon receptor alpha (EqFcεRIα) (13 ng) (prepared as described in Example 7) was added to the wells. (All molecules were biotinylated following standard procedures using biotin-LC-hydrazide). The plate was incubated at RT for two hours and then washed four times using Wash buffer. Alkaline phosphatase (AP)-conjugated streptavidin (50 ng/well) (KPL Labs) was added to each well and the plate incubated at RT for one hour. Control wells were prepared as described above but without the addition of anti-equine IgE mAb or IgE receptors. The plate was washed using Wash buffer, 100 μl of p-nitrophenyl phosphate (Moss, Inc., Pasadena, Md.) added to each well and the plate incubated at RT for one hour. The color reaction was stopped by the addition of 50 μl of 20 mM L-cysteine to each well and the absorbence was read at 405 nM using an ELISA plate reader. The results, with the background values subtracted, are shown in Table 5.

TABLE 5

| | | | |
|---|---|---|---|
| Allergen | H-357 | HuFcεRlα | EqFcεRlα |

Horse #1

| | | | |
|---|---|---|---|
| Black Fly | 970 | 531 | 575 |
| Horse Fly | 1600 | 695 | 1134 |
| D. farinae | 4035 | 3807 | 3942 |

Horse #2

| | | | |
|---|---|---|---|
| Alfalfa | 691 | 448 | 526 |
| Black Ant | 2377 | 1418 | 1539 |
| Horse Fly | 1484 | 732 | 1067 |

Horse #3

| | | | |
|---|---|---|---|
| Black Ant | 2138 | 1719 | 1972 |
| Horse Fly | 450 | 723 | 913 |

Horse #4

| | | | |
|---|---|---|---|
| D. farinae | 732 | 414 | 405 |

Horse #5

| | | | |
|---|---|---|---|
| Beech | 364 | 388 | 351 |

Horse #6

| | | | |
|---|---|---|---|
| Beech | 143 | 140 | 108 |

Horse #7

| | | | |
|---|---|---|---|
| Beech | 305 | 345 | 266 |

The data demonstrates the absorbence obtained when H-357 is used to detect IgE is similar to that observed when either human or equine FcεRIα is used.

### EXAMPLE 9

This example discloses the specificity of anti-equine IgE monoclonal antibody H-357 for horse IgE. The wells of a microtiter plate were coated, at 50 ng/well, with either equine IgE, canine IgE, feline IgE or human IgE in CBC buffer and the plate incubated overnight at 4° C. Excess fluid was removed, 200 μl of Assay buffer added to each well and the plate incubated at room temperature (RT) for one hour. The plate was washed four times using Wash buffer and 50 μl of serially diluted (from 400 to 3.1 ng/ml in Assay buffer) biotinylated anti-equine IgE mAb (biotinylated by standard procedures using biotin-LC-hydrazide) were added to each well. The plate was incubated at RT for two hours and then washed four times using Wash buffer. HRP-conjugated streptavidin (KPL Labs) was added to each well (10 ng/well) and the plate incubated at RT for one hour. Control wells were prepared as described above but without the addition of anti-equine IgE mAb. The plate was washed using Wash buffer, 100 μl of TMB Substrate System added to each well and the plate incubated at RT for 5 minutes. The color reaction was stopped by the addition of 100 μl 1M H₃PO₄ to each well and the absorbence was read at 450 nM using an ELISA plate reader and the results are shown in Table 6. Background levels have been subtracted from the values shown and all values over 100 are considered positive.

TABLE 6

Coating Immunoglobulin

| mAb H-357 | Equine | Canine | Feline | Human |
|---|---|---|---|---|
| (ng/well) | IgE | IgE | IgE | IgE |
| 20 | 2639 | 208 | 3 | 4 |
| 10 | 2567 | 180 | 6 | 0 |
| 5 | 2496 | 161 | 0 | 0 |
| 2.5 | 2077 | 134 | 0 | 0 |
| 1.25 | 1419 | 98 | 0 | 0 |
| .63 | 848 | 61 | 0 | 0 |
| .31 | 484 | 35 | 3 | 2 |
| .16 | 278 | 15 | 1 | 1 |

This example demonstrates mAb H-357 has a much greater specificity for equine IgE than for canine, feline or human IgE.

### EXAMPLE 10

This example discloses the ability of huFc9εRIα to bind surface-bound IgE from various species. This assay was performed using the basic protocol outlined in Example 9 with the exception that biotinylated-huFcεRIα, instead of anti-equine IgE mAb, was used to detect surface-bound IgE. In addition, each well was coated with 100 ng of IgE instead of 50 ng IgE, concentrations of receptor were changed and the incubation time with the huFcεRIα shortened to one hour. Background levels were measured by preparing control wells with all reagents except huFcεRIα. The results of this assay are shown in Table 7. Background values have been subtracted from those shown in the Table.

TABLE 7

Coating Immunoglobulin

| FcεRIα | Human | Canine | Feline | Equine |
|---|---|---|---|---|
| (ng/ml) | IgE | IgE | IgE | IgE |
| 1300 | 1482 | 2286 | 2973 | 2123 |
| 650 | 2109 | 2409 | 3038 | 2108 |
| 325 | 1933 | 2327 | 3007 | 2128 |
| 162.5 | 1896 | 2321 | 2930 | 2058 |
| 81.3 | 2128 | 2307 | 2741 | 1867 |
| 40.6 | 1054 | 2100 | 2732 | 1698 |
| 20.3 | 1039 | 1964 | 2462 | 1344 |
| 10.2 | 1462 | 1911 | 2443 | 1132 |
| 5.1 | 1276 | 1509 | 2147 | 881 |
| 2.5 | 783 | 1050 | 1447 | 556 |
| 1.3 | 574 | 710 | 891 | 330 |

The data demonstrates huFcεRIα is capable of recognizing IgE from humans, canines, felines and equines with equal affinity.

### EXAMPLE 11

This example discloses the isotypes of the various monoclonal antibodies of the present invention. Isotypes were determined using the IsoStrip Kit (mouse monoclonal antibody isotyping kit) (Roche Diagnostics Corporation, Indianapolis, Ind.) according to the manufacturer's instructions. The results of these tests are shown below in Table 8.

TABLE 8

| mAb | |
|---|---|
| Designation | Isotype |
| H99 | IgG₁, k |
| H100 | IgG₁, k |
| H101 | IgG₁, k |
| H102 | IgG₁, k |
| H103 | IgM, k |
| H104 | IgG₁, k |
| H106 | IgG₁, k |
| H107 | IgG₁, k |
| H189 | IgG₂ₐ, k |
| H190 | IgG₁, k |
| H191 | IgG₁, k |
| H192 | IgG₁, k |
| H193 | IgG₂ₐ, k |
| H195 | IgG₁, k |
| H197 | IgG₁, k |
| H198 | IgG₁, k |
| H199 | IgG_{2b}, k |
| H200 | IgG₁, k |
| H201 | IgG₁, k |
| H202 | IgG₁, k |
| H203 | IgG₁, k |
| H204 | IgG₁, λ |
| H205 | IgG_{2b}, k |
| H206 | IgG₂ₐ, k |
| H207 | IgG₁, k |
| H208 | IgG_{2b}, k |
| H219 | IgG₁, k |
| (H187) | |
| H220 | IgG₂ₐ, k |
| (H188) | |

### EXAMPLE 12

This example discloses the ability of EqFcεRIα to bind surface-bound IgE from various species. This assay was performed using the basic protocol outlined in Example 9 with the exception that biotinylated-EqFcεRIα, instead of anti-equine IgE mAb, was used to detect surface-bound IgE. In addition, each well was coated with 100 ng of IgE instead of 50 ng IgE, the concentrations of receptor were changed and the incubation time with the EqFcεRIα shortened to one hour. Background levels were measured by preparing control wells with all reagents except EqFcεRIα. The results of this assay are shown in Table 8. Background values have been subtracted from those shown in the Table 9.

TABLE 9

Coating Immunoglobulin

| FcεRIα | Human | Canine | Feline | Equine |
|---|---|---|---|---|
| (ng/ml) | IgE | IgE | IgE | IgE |
| 1300 | 142 | 1805 | 2612 | 1838 |
| 650 | 173 | 1706 | 2663 | 1777 |
| 325 | 214 | 1733 | 2807 | 1834 |
| 162.5 | 112 | 1562 | 2669 | 1879 |
| 81.3 | 79 | 1657 | 2567 | 1813 |
| 40.6 | 34 | 1464 | 2615 | 1706 |
| 20.3 | 34 | 1115 | 2328 | 1322 |
| 10.2 | 41 | 903 | 2129 | 1151 |
| 5.1 | 36 | 599 | 1653 | 957 |
| 2.5 | 44 | 367 | 1053 | 684 |
| 1.3 | 7 | 222 | 508 | 385 |

The data demonstrates EqFcεRIα binds canine, feline and equine IgE with high affinity but has a much lower affinity for human IgE.

### EXAMPLE 13

This example provides the general protocol for performing the immunoassays of the present invention.

Immediately prior to use, the device is wetted with two drops of wash buffer (TRIS 6.05 g/l; NaCl 8.5 g/l; NaN₃ 20 mg/l; Bovine Serum Albumin (BSA) 2.5 g/l; Tween 20 5.0 ml/l; and pH adjusted with concentrated HCl to 7.5±0.1). The two drops are added one right after the other, and the drops are allowed to absorb before proceeding.

Two drops of sample (undiluted) are then added, one immediately after the other, and the drops are allowed to absorb before proceeding. Two drops of wash buffer are then added, one at a time, and the drops are allowed to absorb before proceeding.

Two drops of biotinylated mouse anti-canine IgE are then added, one immediately after the other, and the drops are allowed to absorb before proceeding. Two drops of wash buffer are then added, one at a time, and the drops are allowed to absorb before proceeding.

Two drops of streptavidin-alkaline phosphatase are then added, one immediately after the other, and the drops are allowed to absorb before proceeding. Two drops of wash buffer are then added, one at a time, and the drops are allowed to absorb before proceeding.

Two drops of enzyme substrate are then added, one immediately after the other, and the drops are allowed to absorb before proceeding. Wait a minimum of 30 seconds, and add four drops of wash buffer are then added, one immediately after the other. Results can be read immediately and are stable for two hours.

In an alternative embodiment of the invention, the detector reagent is another indicator reagent such as immunogold, or a fluorescent or radioactive compound, and the method of reading the results will change appropriately.

Although the invention has been described with reference to the presently preferred embodiments, it should be understood to those skilled in the art that various modifications can be made without departing from the spirit of the invention. Accordingly, the invention is limited only by the following claims.

What is claimed is:

1. A device for detecting allergen-specific IgE in a sample obtained from an animal, said device comprising:
(a) a first member comprising a porous member having a sample receiving area, wherein said porous member is spotted with at least one mixture of allergens and a control spot; and
(b) a support member.

2. The device of claim 1, wherein the mixture of allergens is deposited on one spot of the porous member.

3. The device of claim 1, wherein mixtures of allergens are deposited on multiple spots of the porous member.

4. The device of claim 1, wherein the mixture of allergens is deposited on an area of the porous member separate from the sample receiving area.

5. The device of claim 1, wherein the animal is a dog, cat, horse or human.

6. The device of claim 1, wherein the sample is a bodily fluid obtained from the animal.

7. The device of claim 6, wherein the bodily fluid is whole blood or a component thereof, urine or a mucosal secretion.

8. The device of claim 7, wherein the bodily fluid is whole blood, serum or plasma.

9. The device of claim 1, wherein said device further comprises an absorbent member in capillary communication with the porous first member, wherein said absorbent member is selected to induce flow of liquid through the first member without the use of external means.

10. The device of claim 1, wherein the control spot contains purified IgE.

11. The device of claim 1, wherein said mixture of allergens comprise one or more types of allergens selected from the group consisting of cat allergens, dog allergens, flea saliva proteins, house dust mite allergens, Japanese Cedar pollen, ragweed allergens, ryegrass allergens, meadow fescue, orchard grass, Bermuda grass, sheep sorrel, Kochia, Russian thistle, yellow dock, timothy grass, redtop, birch and blue grass allergens.

12. A method for detecting the presence of allergen-specific IgE in a sample, said method comprising the steps of:
(a) adding the sample to the sample receiving area of the device of claim 1, whereby at least one allergen in the mixture of allergens spotted on the porous member binds to allergen-specific IgE if present in the sample;
(b) contacting the mixture of allergens with a reagent that selectively binds to allergen-specific IgE; and
(c) detecting bound allergen-specific IgE if present in the sample.

13. The method of claim 12, wherein the reagent is labeled.

14. The method of claim 13, wherein the labeled reagent is an anti-IgE antibody or an IgE receptor.

15. The method of claim 14, wherein the labeled reagent is an anti-canine IgE, anti-feline IgE, anti-equine IgE or anti-human IgE monoclonal antibody.

16. The method of claim 12, wherein the sample is a bodily fluid obtained from an animal.

17. The method of claim 16, wherein said bodily fluid is whole blood or a component thereof, urine or a mucosal secretion.

18. A method for prescribing immunotherapy treatment of an animal having an allergic disease, said method comprising the steps of:
(a) obtaining a sample from the animal;
(c) analyzing the sample according to the method of claim 12 to detect the presence of allergen-specific IgE in said sample, wherein the presence of allergen-specific IgE identifies the sample as a positive sample;
(c) analyzing the positive sample to identify one or more allergens responsible for said allergic disease; and
(d) selecting an appropriate immunotherapy for said animal to treat the allergic disease.

19. The method of claim 18, wherein an in vitro immunoassay or an intradermal test is used to analyze the positive sample.

20. A kit comprising the device of claim 1 and ancillary reagents.
